(19) United States
(12) Patent Application Publication
(10) Pub. No.: US 2007/0060882 A1
(43) Pub. Date: Mar. 15, 2007

(54) Method and apparatus for treatment of thrombosed hemodialysis access grafts

(76) Inventor: Michael G. Tal, Woodbridge, CT (US)

Correspondence Address: WELSH & FLAXMAN LLC, 2000 DUKE STREET, SUITE 100, ALEXANDRIA, VA, 22314, US

(21) Appl. No.: 11/598,668

(22) Filed: Nov. 14, 2006

Related U.S. Application Data
(63) Continuation-in-part of application No. 10/947,423, filed on Sep. 23, 2004.
(60) Provisional application No. 60/505,665, filed on Sep. 24, 2003.

(51) Int. Cl. A61M 29/00

(52) U.S. Cl. 604/102.01; 604/915

(57) ABSTRACT

A catheter includes a catheter body with a compliant balloon secured at a distal end of the catheter body. The balloon includes a proximal end and a distal end. At least one infusion aperture is positioned directly adjacent the proximal end of the balloon.

### CROSS REFERENCE TO RELATED APPLICATIONS

This application is a continuation-in-part of U.S. patent application Ser. No. 10/947,423, filed Sep. 23, 2004, entitled “Method and Apparatus for Treatment of Thrombosed Hemodialysis Access Grafts”, which is currently pending, which claims the benefit of U.S. Provisional Patent Application Ser. No. 60/505,665, filed Sep. 24, 2003, entitled “Dialysis Access Thrombectomy Catheter”.

### BACKGROUND OF THE INVENTION

1. Field of the Invention

The present invention relates to the field of interventional radiology. More particularly, the invention relates to a method and apparatus for the reconstruction of a flow path within a vascular conduit. The invention further relates to embolectomy and thrombectomy, including treatment of thrombosed hemodialysis access grafts or fistulas.

2. Description of the Prior Art

Life-sustaining access to hemodialysis is one of the leading causes for hospital admission. More than 80% of the patient population undergoing hemodialysis treatments have a PTFE graft access. However, PTFE graft access only offers an average patency of 20 months after placement.

If one considers that the arterial and venous anatomy is typically sufficient to support three upper extremity grafts, a dialysis patient may expect an average 10 years of permanent access availability from upper extremities; that is, 20 months times six potential grafts. Depending on the age when the kidneys fail, between 23% and 51% of patients will live at least 10 additional years after starting dialysis. If a renal transplant does not become available, many patients will need to resort to peritoneal dialysis or a less preferable hemodialysis access such as a lower extremity graft or a hemodialysis catheter. Some patients may even die because of lack of access. Therefore, efforts to maintain each available permanent hemodialysis access have become a matter of paramount importance.

Thrombosis, or blood clot formation, is the most common cause of hemodialysis access graft failure. Graft thrombosis usually results from venous flow obstruction, or stenosis. The location of the stenosis is most commonly found at the graft-vein anastomosis. A narrowing at this area causes a slow down or obstruction of blood flow, resulting in the formation of the thrombus within the graft. Venous stenosis is present in over eighty-five percent of clotted grafts. The underlying venous anastamotic stenosis must be corrected in order to avoid recurrence of the thrombus.

There are at least three primary interventional radiology methods for percutaneous thrombolysis: Thrombolytic (Urokinase, Stereptokinase, Tissue plasminogen activator CIPA, r-TPA), and other) infusion, pulse-spray pharmacomechanical thrombolysis, and pure mechanical thrombectomy.

Percutaneous thrombolysis is the least invasive treatment option for graft treatment and has rapidly become the preferred method of treatment at most institutions. It is commonly accomplished using mechanical thrombectomy devices that macerate the clot or by using a thrombolytic agent to dissolve the clot. Mechanical thrombectomy devices are expensive and often require capital investment. Thrombolytic agents provide a less expensive treatment option.

Tissue plasminogen activators, also known as TPA, are one of the most commonly used thrombolytic agents for clearing dialysis grafts. The drug is introduced into the clotted graft via an infusion catheter or a needle. TPA has a high affinity and specificity for fibrin, a major component of blood clots. It acts upon the clot by binding to the surface and dissolving it by an enzymatic reaction. The time until clot dissolution is dependent on the length and size of the clot, the amount of drug delivered and method used for drug delivery.

With the “lyse and wait” technique of thrombolysis, TPA or other thrombolytic agent, such as, urokinase or retaplase, is delivered to the graft by a small gauge needle or an infusion catheter. Manual compression is applied to the graft-artery anastomosis during drug administration to ensure targeted drug delivery is restricted to the graft and prevent inadvertent dislodgment of clot into the artery. The procedure is performed without the aid of fluoroscopic guidance. The therapeutic action of the lytic agent typically takes at least one hour depending on the effective distribution of the lytic agent. After clot dissolution, the patient typically is brought into the angiographic suite for fluoroscopic imaging of the graft to identify and visualize residual venous stenosis. Angioplasty of the stenosed segment can then be performed.

With regard to mechanical thrombolysis, several devices are known to have been used. For example, a rotating nitinol basket-like fragmentation cage (Arrow-Trerotola Percutaneous Thrombolytic Device) has been used by crossing 5-F sheaths within a graft and requires only a minute or two to restore flow. In a recent study, fifty-one consecutive patients were treated with the device. In all patients, the device was used to also treat the arterial plug in situ at the arterial anastomosis instead of using a Fogarty catheter to reposition the plug as indicated by the product labeling of the devices. Immediate technical patency was 100% with 6% arterial embolization vs. 2% control. Adjunctive therapy with a Fogarty Adherent Clot catheter was needed in two procedures (4%).

The Amplatz mechanical thrombectomy device (Clot Buster, Microvena Co.), has also been used successfully in dialysis grafts. This 8-French device consists of a gas-driven, high-speed (150,000 rpm) cam that pulverizes the clot. In a randomized series comparing surgical thrombectomy with the device, 89% success was achieved in the device group and 83% in the surgery group. Thirty-day patency was lower with the device (47%) than with surgery (77%). However, residual thrombus may occur with the device, and it cannot be used to treat the arterial plug. Recently, the device has been made available also in a 6-French version. Because the device is not guidewire compatible, a 6-French ID or 8-French ID delivery sheath or an 8-French OD or 6-French OD guiding catheter should be used.

The Hydrolyser catheter (Cordis) uses the Venturi effect to achieve mechanical thrombolysis. The catheter is driven using a conventional angiographic injector. Although testing shows this device was successful in 15/16 instances, five reclotted within 24 hours. Secondary patency was 41% at 6 months. One concern with this device, however, is the amount of blood aspirated during the procedure (50-150 μL), which could be problematic for chronically anemic patients.

The Cragg thrombolytic brush consists of a 6-French brush catheter, and combines mechanical thrombolysis with thrombolytics to shorten procedure time and reduce thrombolytic dose. It is not a purely mechanical thrombolytic approach, but it takes advantage of many principles of mechanical thrombolysis. This 6-French device consists of a nylon brush that rotates at low speed (1,800 rpm.) driven by a single-use detachable motor drive. It is not guidewire compatible. Another similar design is the Castaneda Over-the-Wire Brush (MT1), which is more preferred because of its guidewire compatibility. The brush itself is modified and allows for using the system forward and backward.

U.S. Pat. No. 4,921,484 discloses a device that uses a tubular mesh in a mesh balloon catheter device. Although this design has shown some utility, it does not offer guidewire compatibility. Thus, it may be necessary to use an additional device(s) to steer toward a desired place within a vessel.

Among simpler devices, the Fogarty Arterial Embolectomy Catheter (Baxter Scientific Products, McGaw Park, Ill.) has shown some utility in removing arterial clots. Although the original Fogarty catheters were not guidewire compatible, guidewire compatible Fogarty balloons (Baxter) have recently been made available. Other over-the-wire alternatives include occlusion balloons and PTA balloons to macerate the clots. The basic technique for recanalization of hemodialysis access grafts using these devices often consists of a crossover catheterization requiring, unfortunately, multiple equipment. Specifically, two introducer sheaths and two balloon catheters are used. For dislodgment of an arterial plug or intragraft stenosis, the Fogarty Adherent Clot Catheter (Baxter) has been successfully used in some cases. Another similar alternative is the Fogarty Graft Thrombectomy Catheter (Baxter), which was designed to remove tough, mature thrombus from synthetic grafts. Except for the over-the-wire Fogarty balloon, the other designs have no guidewire compatibility.

Despite many advantages, traditional mechanical thrombolytic devices often exhibit significant drawbacks. Some devices are large (8-French or more) and perform poorly in curved vessels, limiting their use in hemodialysis access. Residual adherent clot is a considerable problem with some mechanical devices. Many devices do not remove the macerated clot and it may be embolized into the lungs. Some mechanical devices cause damage to the endothelial lining of a fistula. A great number of the available devices cannot be used over-the-wire.

Another method was recently described in which access is achieved toward the venous and arterial anastomosis and an occlusion balloon catheter is inflated at the arterial anastomosis site. While the balloon is inflated, a large quantity (approximately 40-60 cc) of saline is injected into the graft through the sheath, “washing” the residual clot away. The presence of the balloon is “protecting” the artery from embolization of clot into it, a major and infrequent complication. The occlusion balloon is then inflated in the arterial anastomosis site or adjacent to it. Again, infusion of saline or contrast material or thrombolytic drugs can be injected. The technique is working very well, however, the whole length of the graft cannot be cleared or visualized.

With the foregoing apparatuses in mind, a preferred current technique for comprehensive shunt cleansing begins with inserting a needle through the skin and into the shunt. A small wire is then inserted through the needle and the tactile sensation transmitted by the wire is used in determining whether the wire is in the shunt. The skin site is then inspected with X-ray to determine the position of the wire and whether it is within the shunt, the needle is removed when the wire is determined to be in the shunt interior, a small catheter is placed over wire with the discharge orifice within the shunt and the wire is removed leaving the catheter with its discharge end within the shunt.

The larger wire is then inserted through the catheter into the shunt interior and the catheter is removed. The next step involves inserting a sheath over the larger wire and into the shunt. A balloon catheter is then advanced into the venous anastomosis and the balloon is inflated to crush the venous anastomosis and open the shunt-vein juncture. Thereafter, the balloon and wire are removed, a second sheath is inserted between the position of the first sheath insertion and shunt-vein juncture, into a clean shunt region, and the clot is macerated and eradicated either mechanically or pharmacologically.

A balloon is then pushed into position within arterial anastomosis at the artery-shunt juncture and the balloon is inflated and pulled back, eradicating the arterial plug and removing the platelet plug and residual arterial anastomosis from the shunt-artery juncture by pulling on the balloon.

Unfortunately, injection of a contrast material into the graft cannot be safely performed before flow in the graft is reestablished. In some cases, flow cannot be established and the operator cannot tell what is the cause for the lack of success. After flow is reestablished, the operator may eradicate additional visualized stenosis. The final step is that of removing the balloon, wire and the sheath.

As those skilled in the art will appreciate, the prior art techniques relating to the treatment of a thrombosed hemodialysis access graft or fistula exhibit various shortcomings. In particular, current techniques offer no safe mechanism for the application of thrombolytic solutions and contrast solutions within the occluded graft due to concerns relating to the migration of clots into the arterial system. As such, thrombolysis and imaging of the graft must be achieved utilizing additional steps and procedures. This is undesirable. The present invention overcomes the shortcomings of the prior art by providing an effective and reliable method and apparatus for the reconstruction of a flow path within a vascular conduit. It also provides a way to safely inject contrast material and thrombolytic drugs, as well as saline or any other fluid to flush a clot from an occluded graft prior to restoration of flow.

### SUMMARY OF THE INVENTION

It is, therefore, an object of the present invention to provide a catheter having a catheter body with a compliant balloon secured at a distal end of the catheter body. The balloon includes a proximal end and a distal end. At least one infusion aperture is positioned directly adjacent the proximal end of the balloon.

It is also an object of the present invention to provide a catheter wherein the catheter body includes a first lumen in fluid communication with the interior of the balloon allowing the balloon to be inflated and deflated under the control of a syringe coupled to the first lumen and a second lumen maintained in fluid communication with the at least one infusion aperture located directly adjacent the proximal end of the balloon.

It is also another object of the present invention to provide a catheter wherein the balloon is latex.

It is also a further object of the present invention to provide a catheter wherein the balloon includes an elongated body having a distal portion adjacent the distal end of the balloon and a proximal portion adjacent to the proximal end of the balloon, and compliance of the balloon changes from its distal end to its proximal end.

It is still a further object of the present invention to provide a catheter wherein the distal portion of the balloon is more compliant than the proximal portion of the balloon and, therefore, inflates first.

It is yet a further object of the present invention to provide a catheter wherein the balloon is substantially cylindrical with a constant diameter along its length.

It is another object of the present invention to provide a catheter wherein the balloon has a length of approximately 3 cm.

It is still another object of the present invention to provide a catheter wherein the balloon is hourglass shaped.

It is yet another object of the present invention to provide a catheter wherein the balloon is frustoconically shaped, and the proximal portion of the balloon has a larger diameter than the distal portion of the balloon.

It is also an object of the present invention to provide a catheter wherein the proximal end of the balloon is designed to extend proximally and slightly cover the infusion apertures.

It is also another object of the present invention to provide a catheter including a catheter tip provided at the distal end of the catheter body at a position distal to the distal end of the balloon, wherein the catheter tip is provided with a relatively sharp but soft distal end and includes a hydrophilic surface.

It is also a further object of the present invention to provide a catheter wherein the catheter tip includes a taper optimizing reduced drag as the catheter tip is moved through a clot.

It is a further object of the present invention to provide a catheter wherein the catheter tip is curved allowing steering across an arterial anastomosis.

It is also an object of the present invention to provide a catheter including two infusion apertures that are diametrically opposed.

It is another object of the present invention to provide a catheter including radiopaque markers at the distal end of the catheter body.

It is still a further object of the present invention to provide a catheter wherein the balloon is radiopaque.

It is yet another object of the present invention to provide a catheter wherein the at least one infusion aperture is substantially closed until such a time that adequate pressure is applied for opening the at least one infusion aperture.

It is also an object of the present invention to provide a catheter wherein the catheter is approximately 40 cm to approximately 60 cm long.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIG. 1 is a side schematic view of an inflated balloon catheter within a vessel in accordance with the present invention.

FIG. 1A is a side view of the balloon catheter when the balloon is inflated but not confined in a vessel.

FIG. 2 is a cross sectional view of a balloon catheter shown in FIG. 1 along the line II-II.

FIG. 2A shows various alternate cross sectional profiles that may be used in accordance with the present invention.

FIG. 3 is a detailed schematic view of the distal end of the inflated balloon catheter while within a vessel.

FIGS. 4 and 5 are detailed schematic views of the distal end of balloon catheters, when the balloon is inflated but not confined in a vessel, in accordance with various alternate embodiments.

FIG. 6 is a detailed schematic view of the distal end of a balloon catheter, while the balloon is inflated within a vessel, in accordance with an alternate embodiment of the present invention.

FIGS. 7 to 13 show the steps associated with treatment of thrombosed hemodialysis access grafts or fistulas in accordance with the present invention.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for the claims and as a basis for teaching one skilled in the art how to make and/or use the invention.

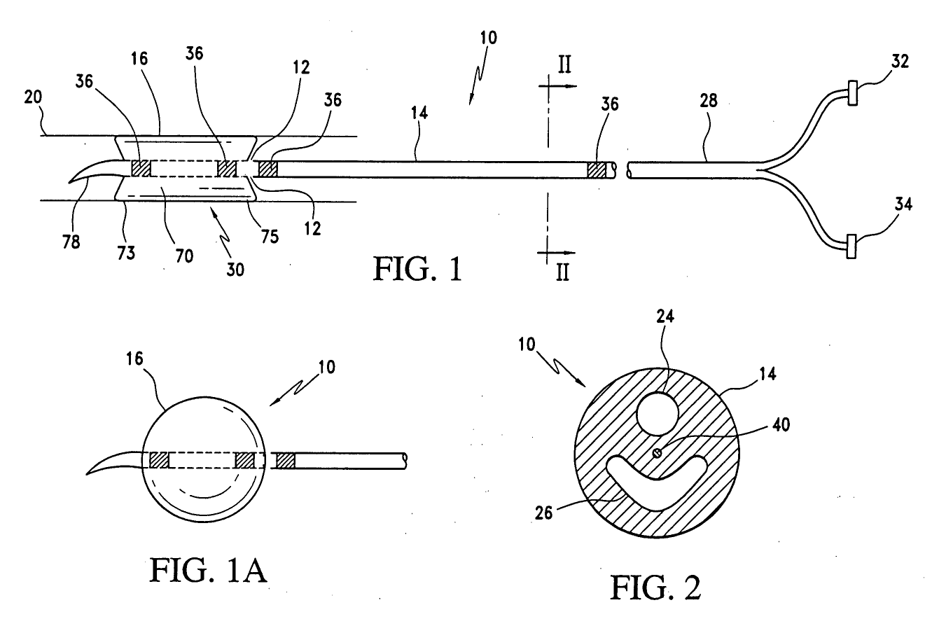

With reference to FIGS. 1, 2 and 3, a balloon catheter 10 with infusion apertures 12 positioned directly adjacent the proximal end 75 of the balloon 16 for the injection of thrombolytic agents, contrast materials and/or any other fluid such as saline or a combination of fluids is disclosed. The present balloon catheter 10 is preferably designed for use in dialysis access declotting, although those skilled in the art will appreciate that it may be used for a variety of applications, such as, dialysis fistula or a native artery or vein. In accordance with a preferred embodiment of the present invention, the catheter 10 is approximately 40 cm to approximately 60 cm long, although those skilled in the art will appreciate other lengths may be used without departing from the spirit of the present invention.

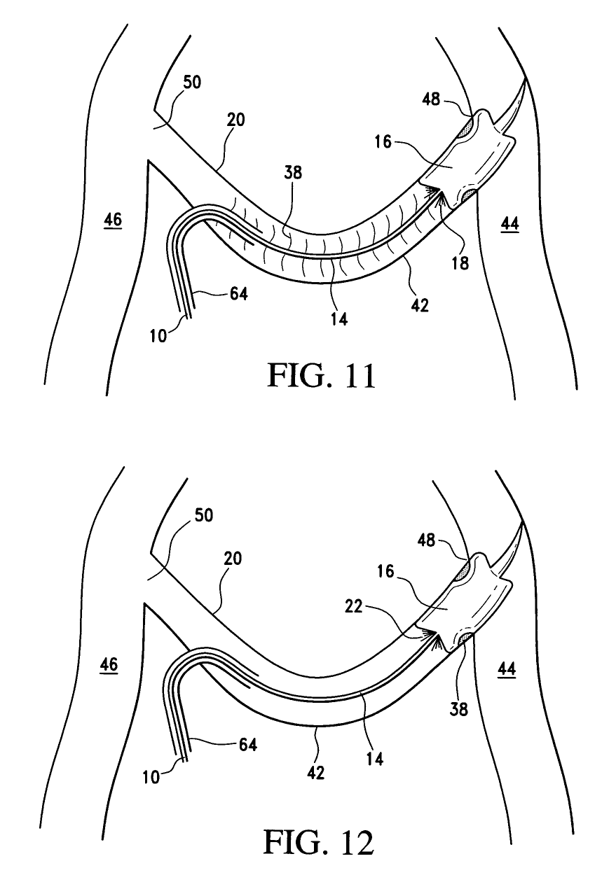

The present balloon catheter 10 is designed for performing various functions during dialysis access (graft or fistula) procedures. For example, the balloon catheter 10 is designed for injecting thrombolytic agents 18 into a graft 42 or fistula, either via pulse-spray or by instillation (see FIG. 11). This is accomplished with the balloon 16 inflated (if the catheter 10 is directed towards the arterial side 44 of the vessel, for example in accordance with a preferred embodiment of the present invention, the shunt 20 composed of a graft 48 as shown in FIG. 11) or with the balloon 16 deflated (if the catheter 10 is directed toward the venous side 46 of the shunt 20). Although the present catheter is disclosed herein particularly for use in conjunction with a vessel such as a shunt composed of a graft, those skilled in the art will appreciate the present catheter may be used in conjunction with various body vessels without departing from the spirit of the present invention.

The balloon catheter 10 is also adapted for imaging of the graft 42 during thrombolysis or mechanical thrombectomy and visualization of any residual clot. This is accomplished by injecting a contrast material 22 while the balloon 16 is inflated along the arterial side 44 of the shunt 20 (see FIG. 12). As those skilled in the art will appreciate, no current technique or device offers a mechanism for visualizing the graft prior to reestablishment of flow. The balloon catheter 10 is further adapted for flushing a clot 38 from the graft 42 to reestablish flow. This is accomplished by inflating the balloon 16 in the graft 42 adjacent to the arterial anastomosis 48 and injection a volume of saline (approximately 30-60 cc) into the graft 42. The saline could be mixed with contrast, a thrombolytic agent or any other medication or a combination of thereof.

When flow is restored in the graft 42, the balloon catheter 10 may also assist in imaging of the whole graft 42, including the arterial anastomosis 48 with a single injection. This is achieved by inflating the balloon 16 at the venous anastomosis 50 and applying a contrast material through the infusion apertures 12. Through the utilization of this technique there is no need to occlude the graft with a Kelley clamp, operator finger or with an angioplasty balloon. Techniques such as those described above can be used with the angioplasty procedures of the venous anastomosis, in addition to thrombectomy/lysis.

As those skilled in the art will appreciate, the present balloon catheter 10, when employed in accordance with the procedure outlined below will replace the Fogerty thrombectomy balloon and Cragg-MacNamara or Angiodynamics multi-side hole infusion catheters. The balloon catheter 10 advantageously allows for a rapid clot removal procedure with visualization of the graft 42 and working environment, replaces dual catheters employed in accordance with the prior art techniques with a single catheter and makes the procedure safer for the patient by reducing time of the procedure and risk of arterial embolization and for the operator, reducing radiation exposure. Although those skilled in the art will appreciate a variety of uses for the present balloon catheter 10, the balloon catheter 10 is preferably used for thrombolytic declotting (TPA, rTPA and Urokinase) and mechanical declotting, whether with the Angiojet, Trerotola, or any other mechanical thrombectomy devices.

More particularly, the balloon catheter 10 includes a longitudinally extending catheter body 14 having a balloon 16 secured at a distal end 30 of the catheter body 14. The distal balloon 16 is formed over the catheter body 14 in a traditional manner to substantially seal an arterial anastomosis 48 as discussed below in substantial detail. It is important to understand the distal balloon 16 must be occluding the arterial anastomosis 48 prior to injection of thrombolytics or contrast materials. As will be appreciated based upon the following disclosure, the balloon 16 must reliably stay in place during injection of thrombolitics and/or contrast agents. The injection of any fluid or medication might increase the pressure inside the graft 42. This increase in pressure could potentially push the balloon 16 back into the adjacent artery if the balloon 16 is not properly anchored in place. If the balloon 16 migrates into the artery during injection, it is likely to cause clot material to migrate into the artery as well, potentially causing a serious complication. If by mistake the distal balloon 16 is inflated at the venous anastomotic site, the result would be migration of clot into the artery and arterial embolization, the exact complication the present invention aims to prevent.

In accordance with a preferred embodiment, and with reference to FIGS. 1, 2 and 3, the catheter body 14 includes at least two lumens 24, 26 respectively maintaining fluid communication between the proximal end 28 of the catheter body 14 and the distal end 30 of the catheter body 14 where the infusion apertures 12 and distal balloon 16 are positioned. The first lumen 24 is maintained in fluid communication with the interior of the distal balloon 16 allowing the balloon 16 to be inflated and deflated under the control of a syringe (not shown) coupled to the first lumen 24 via a port 32 located at the proximal end of the first lumen 24. The first lumen 24 must be shaped and dimensioned to permit the free flow of an inflation medium during inflation and deflation of the distal balloon 16.

The second lumen 26 is maintained in fluid communication with the infusion apertures 12 positioned directly adjacent the proximal end 75 of the distal balloon 16. The second lumen 26 allows for the passage of fluids through the catheter body 14 for passage through the infusion apertures 12 for reasons discussed below in greater detail. As with the first lumen 24, the second lumen 26 is provided with a port 34 at its proximal end for the application of the thrombolytic agent 18, contrast material 22 and/or other solutions. As with the first lumen 24, the second lumen 26 must be shaped and dimensioned to permit the free flow of thrombolytics, contrast materials and/or other solutions to the infusion apertures 12. In accordance with a preferred embodiment of the present invention, the second lumen 26 is relatively large allowing for rapid infusion of the treatment site through the flow of thrombolytic agent therethrough, without the creation of substantial back-pressure that might result if the lumen were too small to accommodate a substantial flow of thrombolytic agent. In accordance with a preferred embodiment, the catheter 10, is a 5 or 6 French, although those skilled in the art will appreciate the size may be varied to accommodate various needs without departing from the spirit of the present invention.

As discussed below, the balloon 16 of the present catheter 10 is not used for dilatation purposes and is formed to function as an occlusion balloon. With this in mind, the distal balloon 16 is constructed to be highly compliant and may be composed of one or more layers of expandable material, such as, polyurethane, radiopaque polyurethane material, thermoplastic polyurethane elastomers, aliphatic polyurethanes, aromatic polyurethanes, styrene-ethylene-butylene-styrene (SEBS) block copolymer, thermoplastic elastomers, low-density polyethylene, polyethylene terephthalate, polyethylene terephthalate glycol, silicone, copolymer of polyurethane and silicone, natural rubber, synthetic rubber, thermoplastic polyamide, nylon, latex, polyethylene, polyisoprene, polyisobutylene, thermoplastic elastomers, an elastomeric material, or combinations thereof. In accordance with a preferred embodiment of the present invention, a latex balloon 16 has been chosen for use in accordance with a preferred embodiment based upon the compliance and softness of the material. By utilizing a latex balloon 16, it is contemplated less vessel wall trauma will be encountered as the balloon 16 is moved through the vessel, for example, shunt 20, in the manner discussed below in greater detail.

In accordance with a preferred embodiment, the balloon 16 includes an elongated body 70 having a distal portion 72 adjacent the distal end 73 of the balloon 16 and a proximal portion 74 adjacent to the proximal end 75 of the balloon 16. The compliance of the balloon 16 changes from its distal end 73 to its proximal end 75. The distal portion 72 of the balloon 16 is preferably more compliant than the proximal position 74 thereof and would, therefore, inflate first. This protects against inadvertent migration of clot into the artery while inflating the balloon 16, if the proximal portion 74 of the balloon 16 were to inflate first. The different compliance between the proximal portion 74 and the distal portion 72 also allows the stiffer part of the balloon 16 to be securely coupled in the graft, providing strong occlusion.

Referring to FIGS. 1 and 3, and in accordance with a preferred embodiment of the present invention, a balloon 16 for use in accordance with the present invention is disclosed. In accordance with one embodiment, the balloon 16 is substantially cylindrical with has constant diameter along its length. In accordance with a preferred embodiment, the balloon 16 has a length of approximately 3 cm. Shorter balloons commonly used in thrombectomy procedures might inadvertently get pushed back into the artery during injection of contrast agent and/or thrombolytic agent into the graft. In fact, they are commonly not designed to occlude the vessel, but are designed to pull the clot. While pulling the clot, they scrape the wall of the vessel. This might be less important in a graft, but is very important in fistula and other native vessels. As a result, the longer balloon 16 used in accordance with the present invention overcomes these limitations of shorter balloons traditionally employed.

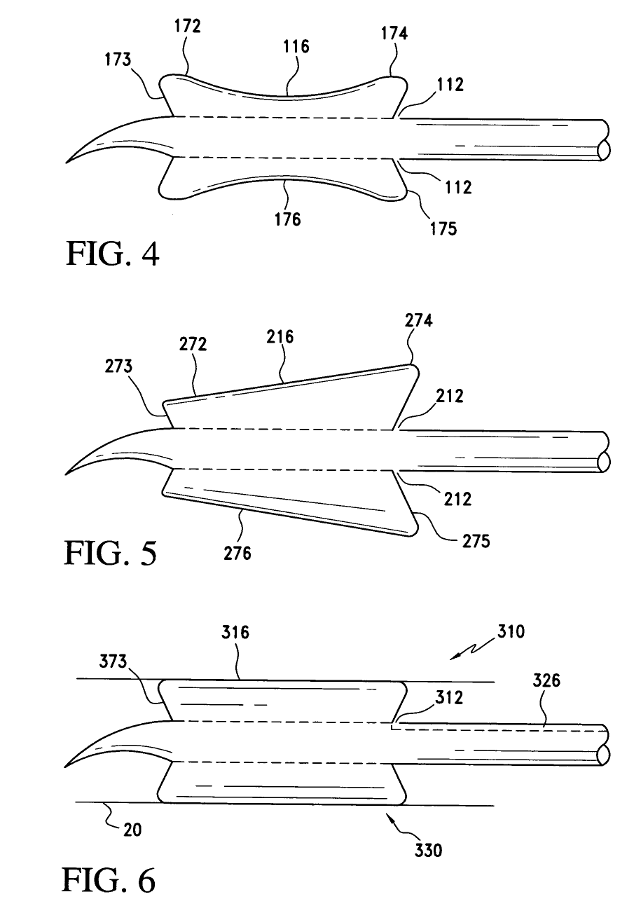

The greater length offers more contact area with the vessel wall, which ultimately increases the frictional resistance of balloon 16 to movement relative to the vessel wall, increasing the balloon's ability to maintain its position while the thrombolytic agent and/or contrast agent is injected within the site. Although the balloon 16 in accordance with a preferred embodiment is sausage shaped, in accordance with alternate embodiments the balloon 116, 216 may be hourglass shaped (see FIG. 4) or frustoconically shaped (see FIG. 5). The cylindrical balloon 16 is shown with reference to FIG. 3, and includes a proximal end 75, a distal end 73 and a central portion 76. As discussed above, the proximal and distal portions 74, 72 of the balloon 16 are preferably constructed with different material characteristics enhancing the balloon's ability to function in accordance with the present invention.

Referring to FIG. 4, and with reference to the hourglass shaped balloon 116, the balloon 116 is hourglass shaped, and includes a proximal portion 174 at the proximal end 175, a distal portion 172 at the distal end 173 and a central portion 176. The distal portion 172 and proximal portion 174 have diameters that are substantially larger than that of the central portion 176. As with the prior embodiment, the infusion apertures 112 are located directly adjacent to the proximal end 175 of the balloon 116. As with the embodiment disclosed with reference to FIGS. 1 and 3, the hourglass shaped balloon 116 exhibits increased compliance along the distal portion 172.

In accordance with another preferred embodiment, and with reference to FIG. 5, it is contemplated the balloon 216 may be constructed with a frustoconical shape and taper to a larger diameter adjacent the proximal end 275 thereof for positioning within the graft. As with the other embodiments, this balloon 216 includes a proximal portion 274 at the proximal end 275, a distal portion 272 at the distal end 273 and a central portion 276. The proximal portion 274 has a diameter that is substantially larger than the distal portion 272, and the diameter tapers as the balloon extends from the proximal end 275 to the distal end 273. The use of a balloon 216 having a larger diameter adjacent its proximal end 275 will result in a greater resistance to movement of clot material around the balloon 216 and into the artery. As with the prior embodiment, the infusion apertures 212 are located directly adjacent to the proximal end 275 of the balloon 216. As with the embodiment disclosed with reference to FIGS. 1 and 3, the frustoconical shaped balloon 216 exhibits increased compliance along the distal portion 272.

Although the following discussion references only the balloon disclosed with reference to the embodiment disclosed in FIGS. 1, 2 and 3, the disclosure herein applies equally as well to the alternate balloon constructions disclosed with reference to FIGS. 4 and 5. The balloon 16 used in accordance with the present invention is also preferably stronger than balloons used in conjunction with conventional embolectomy procedures as they are rather soft, so as to not damage the vessel when pulling the clot. Although the balloon 16 of the present catheter 10 is relatively soft, it does not fully rely on pulling and, therefore, may be constructed with much better strength characteristics.

The use of a long balloon 16, with different compliance along the length of the balloon 16, in particular, being more compliant distally than proximally, facilitates anchoring of the balloon 16 in the anastomosis and prevention of balloon 16 slippage during the procedure. In addition to assisting in anchoring the balloon 16 more firmly in the anastomosis, the use of thicker and/or stiffer balloon material (that is, less compliant) at the proximal portion 74 of the balloon 16 further prevents clot material from slipping distally past the balloon 16 and into the arterial region during insufflations of the balloon 16 or injection of fluid to the proximal infusion apertures 12 as described in the present invention. In addition, the provision of a relatively compliant central portion 76 and distal portion 72 results in a balloon 16 that is better adapted to achieve a desirable seal as the balloon 16 is inflated within the anastomosis. The relatively compliant central portion 76 also may trap additional wall clot while the catheter 10 and clot are withdrawn from the vessel. A distal compliant and proximal less compliant balloon 16 makes migration of clot into the artery during insufflation of the balloon 16 and injection less likely.

A catheter tip 78 is provided at the distal end 30 of the catheter body 14 at a position distal to the distal end 73 of the balloon 16. The catheter tip 68 is preferably approximately 1 cm to 2 cm in length. However, and as those skilled in the art will appreciate, the catheter tip could be formed with different lengths for different balloon sizes. Its construction is ultimately important to the functionality of the present catheter 10 in accordance with the procedure described below in greater detail. In particular, it is important that the catheter 10 be able to move through the clot without pushing the clot forward and into the artery. The catheter tip 78 should also be constructed to reduce the potential for vessel trauma as the catheter 10 is moved through the vessel. The catheter tip 78 should be soft so as to prevent trauma to the native artery when the balloon 16 is advanced through the arterial anastomosis. It should also taper towards the distal end 80 of the catheter tip 78 thereof to further reduce the chances of clot migration while advancing the catheter 10.

With this in mind, the catheter tip 78 is provided with a relatively sharp and soft distal end 80 and potentially includes a hydrophilic surface 82. Ease of movement through the clot is further enhanced by providing the catheter tip 78 with a taper optimizing reduced drag as the catheter tip 78 is moved through the clot. While the catheter tip 78 is relatively sharp, it is constructed from a soft material which will readily give when it contacts a vessel wall or other tissue structure. In accordance with a preferred embodiment, the catheter tip 78 has a length of approximately 2 cm, although those skilled in the art will appreciate of tip lengths may certainly be employed without departing from the spirit of the present invention.

Referring to the various figures, and in accordance with a preferred embodiment the catheter tip 78 is curved allowing improved steering of the balloon catheter 10 across the arterial anastomosis. In accordance with a preferred embodiment, the catheter tip 78 extends about an arc of approximately 30° to approximately 45° with a radius of curvature of approximately 1 cm or less.

As briefly discussed above, proximal to the distal balloon 16 are infusion apertures 12 through which thrombolytic agent 18, contrast material 22 and/or saline or any other medication or a combination of thereof is delivered to the treatment site in a manner discussed below in greater detail. In accordance with a preferred embodiment, the plurality of infusion apertures 12 are positioned directly adjacent to the proximal end 75 of the balloon 16 such that they are diametrically opposed. This orientation results in the best performance. Although two diametrically opposed infusion apertures are disclosed in accordance with a preferred embodiment, it is contemplated that a single infusion aperture or more than two infusion apertures may be employed without departing from the spirit of the present invention.

In accordance with a preferred embodiment, the infusion apertures 12 are directly adjacent the balloon 16. In fact, it is important the infusion apertures 12 are immediately directly adjacent to the balloon 16, because by positioning the infusion apertures 12 directly adjacent the balloon 16, no flow of the thrombolytic agent will go toward the balloon 16 (that is, distally of the infusion area), minimizing the possibility the thrombolytic agent will push the clot toward the distal end of the balloon catheter 10 and ultimately into the artery. Those skilled in the art will understand use of the term “directly adjacent” is meant to indicate the edge 84 of the infusion aperture 12 is in contact with or minimally spaced from the proximal end 75 of the balloon 16 so that there is a minimum amount of space between the infusion aperture 12 and the proximal end 75 of the balloon 16.

In fact, and in accordance with a preferred embodiment of the present invention, the balloon 16 is designed to extend proximally and slightly cover the infusion apertures 12, while inflated in a tubular structure such as a vessel or a graft. In particular, the balloon 16 is shaped to expand as shown in FIG. 1A and assume a spherical or oval configuration when not in a vessel, but is constructed to assume the overlapping relationship when inflated within a vessel as a result of the inward bias of the vessel wall acting upon the balloon 16 (see FIGS. 1 and 3). As a result, the balloon 16 will push the clot material away from the artery and the balloon 16, and the infusion apertures 12 will direct the thombolytic agent proximally away from the balloon 16 and the artery. More particularly, the balloon 16 is constructed such that the wall 86 thereof adjacent the proximal end 75 of the balloon 16 extends in a proximal direction. As a result a line extending perpendicularly from the surface of the catheter body 14 at the infusion aperture 12 will intersect with the wall 86 at the proximal end 75 of the balloon 16.

In accordance with a preferred embodiment, the two opposed infusion apertures 12 are circular holes located directly in contact with the proximal end 75 of the balloon 16. In accordance with a preferred embodiment of the present invention, the infusion apertures 12 are relatively small for creating pressure during the application of the thrombolytic agent 18. In fact, the infusion apertures 12 may be formed in such a way that they are substantially closed until such a time that adequate pressure is applied for opening the infusion apertures 12 and permitting the thrombolytic agent 18 (or contrast material 22 or other solution) to be sprayed therefrom at a relatively high pressure. The spraying of the thrombolytic agent 18 in this way creates a mechanical cleansing action that complements the chemical action of the thrombolytic agent 18. A variety of thrombolytic agents are known to those skilled in the art and various thrombolytic agents may be employed without departing from the spirit of the present invention.

As with the various balloon constructions and other variations discussed above, alternative embodiments of the balloon catheter are contemplated in keeping within the spirit and scope of the present invention. For example, while two infusion apertures are disclosed in accordance with a preferred embodiment of the present invention, FIG. 6 illustrates a balloon catheter 310 constructed with a single infusion aperture 312. In fact, it has been found that a single infusion aperture 312 directly adjacent the proximal end 375 of the distal balloon 316 results in ideal imaging characteristics. In addition to the reason discussed above, the positioning of the infusion aperture 312 proximally and directly adjacent the distal balloon 316 results in a flow of contrast material that makes imaging with the present balloon catheter 310 highly effective. More specifically, by positioning the single infusion aperture 312 proximally and directly adjacent the distal balloon 316, the contrast material is able to opacify the entire graft 42 with a single injection of contrast material. The single aspirating aperture or hole 312 may be formed to create a spray which is proximally angled instead of perpendicular to the axis of the lumen 326 to create a jet effect into the graft. As with the prior multiple infusion aperture embodiment, the infusion aperture 312 is directly adjacent the balloon 316, preferably, directly in contact with the balloon 316. By positioning the infusion aperture 312 directly adjacent the balloon 316 no flow of the thrombolytic agent will go toward the balloon 316 (that is, distally of the infusion area, minimizing the possibility that the thrombolytic agent will push the clot toward the distal end 330 of the balloon catheter 310 and ultimately into the artery).

Regardless of whether a single infusion aperture is employed or multiple infusion apertures are employed, the balloon catheter 10 includes a stiff shaft 40 that is torqueable and extends to the straight or angular, flexible and soft catheter tip 78 allowing the catheter 10 to be steerable. In accordance with a preferred embodiment, the catheter 10 is not provided with an actual steering mechanism as the angular, flexible catheter tip 78 is simply used in getting the catheter 10 to the desired treatment site. The combination of the long catheter tip 78 and the stiff shaft 40 provide a catheter 10 that may be steered through the vessel without collapsing while penetrating through the clot material. The catheter tip 78 also is preferably provided with radiopaque markings 88.

As mentioned above, the catheter 10 is constructed with a dual lumen structure. The use of the dual lumen construction contributes to the stiffness of the catheter 10 in that the septum between the two lumens might add some stiffness.

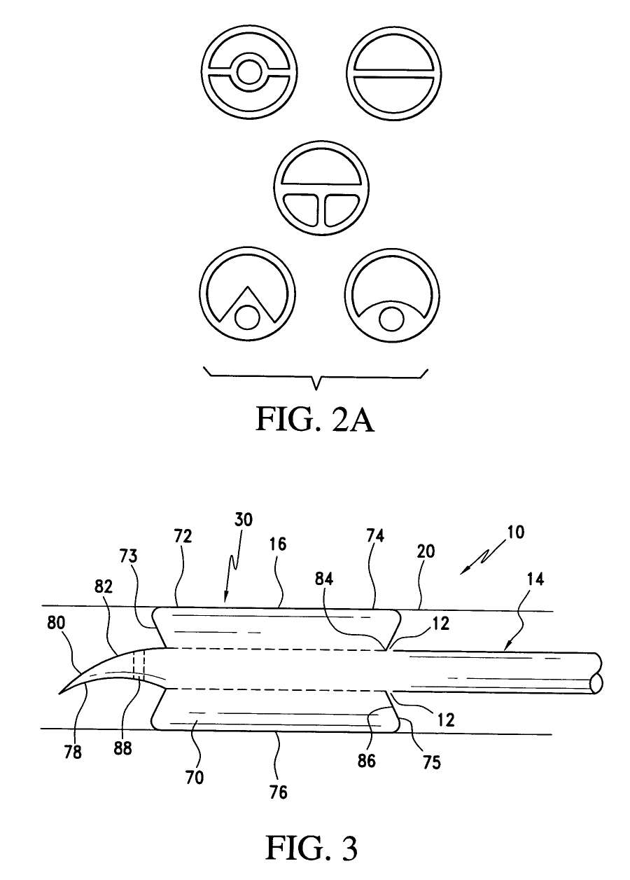

While specific balloon catheter constructions are disclosed above in accordance with a preferred embodiment of the present invention, still other variations on the balloon catheter construction may be employed without departing from the spirit of the present invention. For example, the balloon catheter may be constructed as an over-the-wire (0.018-0.038″) balloon catheter and, therefore, be constructed with three lumens. In addition, although an exemplary cross sectional profile of the two lumen balloon catheter 10 of FIG. 1 is shown in FIG. 2, a wide variety of cross sectional profiles may be employed in accordance with the embodiments of FIGS. 1 and 2 without departing from the spirit of the present invention. Exemplary alternate cross sectional profiles are shown in FIG. 2A.

Radiopaque markers 36 are also positioned at various positions along the treatment region, or the balloon 16 itself may be radiopaque. The positioning of the various radiopaque markings 36 is chosen to assist in visualizing the catheter and treatment area and confirming the positioning of the balloon catheter 10 within the anastomosis. In accordance with a preferred embodiment, one marker 36 is in the distal end 73 of the balloon 16, another marker is in the proximal end 75 of the balloon 16. Additional markers 36 will be positioned just proximal to the proximal infusion apertures 12. The structure of catheter 10 is advantageous in that it will completely contain the thrombolytic agent 18 and all disrupted clot material 38 proximally of the distal balloon 16. Aspiration means may also be provided, e.g., through an additional lumen within the catheter body 14, in order to withdraw materials from the treatment region.

The present balloon catheter may also be provided with a hydrophilic coating enhancing its ability to perform in accordance with the present invention. More particularly, and as discussed above in accordance with a preferred embodiment, the hydrophilic coating is applied to the catheter tip 78 of the catheter 10, possibly also to the balloon and the catheter shaft. This will allow safer advancement of the catheter through clotted vessel or graft reducing the risk of pushing clot distally.

It is also contemplated the catheter may be provided with a distal end hole at the end of the catheter. In such an instance, an occlusion wire would be used to occlude the distal hole after “over-the-wire” placement and prior to injection of fluid into the graft/fistula. The distal end hole would be formed such that the distal elongated catheter tip of the balloon catheter is hollow with a small hole at the tip. This would allow passage of wire through it if necessary. Another possible embodiment would be that the wire itself would be occlusive and that injection of the fluid will be done into the same lumen with the wire, possible through a valved Y-adaptor, with the fluid flowing adjacent to the wire and out of the infusion apertures.

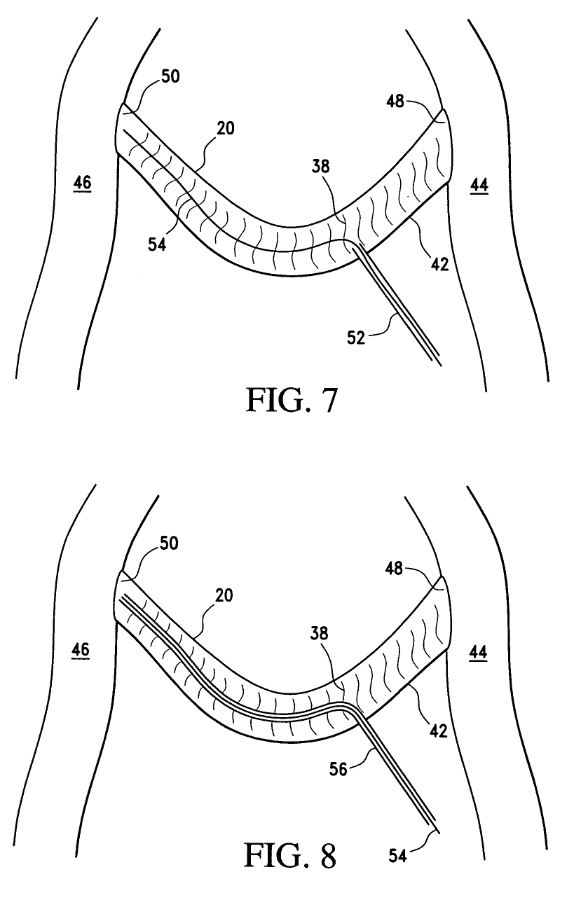

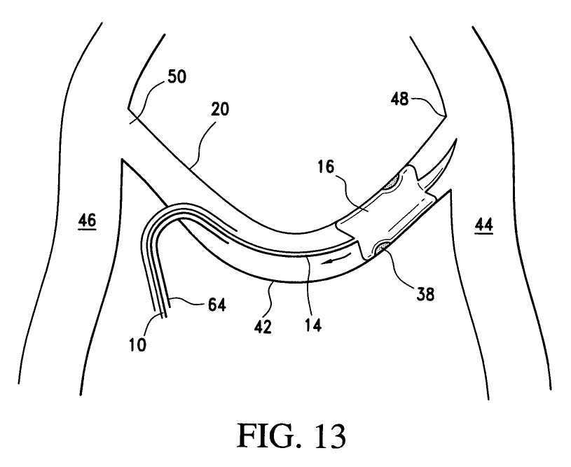

With reference to FIGS. 7 to 13, and in accordance with a preferred embodiment of the present invention, the present shunt cleansing procedure begins with the insertion of a needle 52 through the skin and into the shunt 20. Next, a small wire 54 is inserted through the needle 52, tactile sensation transmitted by the wire 54 is employed in determining whether the wire 54 is in the shunt 20 and the skin site is inspected with X-ray to determine the position of the wire 54 and whether it is within the shunt 20.

The needle 52 is then removed when the wire 54 is determined to be in the shunt 20 interior and a small catheter 56 is placed over wire 54 with the discharge orifice within the shunt 20 (see FIG. 8). The wire 54 is then removed.

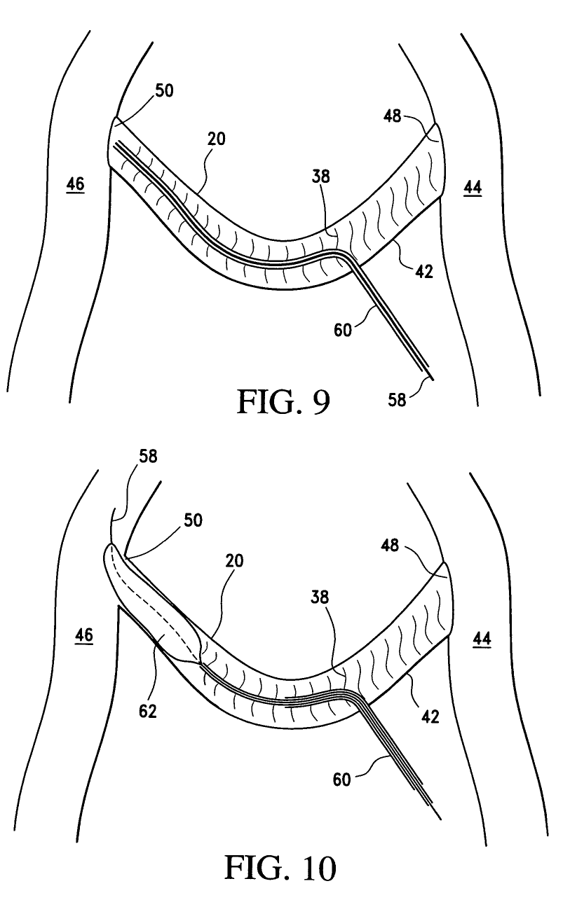

Referring to FIGS. 8 and 9, after the wire 54 is removed, a larger wire 58 is inserted through the catheter 56 into the shunt 20 interior, the catheter 56 is removed and a sheath 60 is inserted over the larger wire 58 and into the shunt 20. A dilatation balloon catheter 62 is then advanced into the venous anastomosis 50 and the balloon catheter 62 is inflated to crush the venous anastomosis 50 and open the shunt-vein juncture (see FIG. 10). The balloon catheter 62 and wire 58 are then removed and a second sheath 64 is inserted between the first sheath 60 insertion and the shunt-vein juncture into a clean shunt region (see FIG. 11).

Thereafter, and with reference to FIG. 11, a thrombectomy balloon catheter 10 in accordance with the present invention as disclosed with reference to FIGS. 1, 2, 3 is pushed into position within arterial anastomosis 48 at the artery-shunt juncture and the distal balloon 16 is inflated to substantially seal the arterial anastomosis 48. As discussed above, it is important to understand that the distal balloon 16 must be occluding the arterial anastomosis 48 prior to injection of thrombolytics or contrast materials. If by mistake the distal balloon 16 is inflated at the venous anastomotic site, the result would be migration of clot into the artery and arterial embolization, the exact complication we aim to prevent with this invention. A thrombolytic agent 18 is then injected through the second lumen 26 and the infusion apertures 12. The thrombolytic agent 18 chemically destroys the clot 38 while the force of the spray created by the application of the thrombolytic agent 18 through the infusion apertures 12 mechanically disrupts the clot 38. By applying the thrombolytic agent 18 with the distal balloon 16 inflated at the arterial anastomosis 48, the possibility of the clot 38 moving into the artery is prevented and there is no need to worry about the migration of the clot into the arterial system and the resulting complications.

The application of the thrombolytic agent 18 can be followed by aspiration of clot or mechanical thrombectomy and can be later followed by the injection of saline to flush the residual clot. Referring to FIG. 12, this is then followed by the application of a contrast material 22 through the second lumen 26 and the infusion apertures 12 to visualize the graft 42 and residual clots or stenosis, if any. As long as the balloon is insufflated at the arterial anastomosis, the sequence of the above procedures can be changed based on operator preference. The application of contrast material 22 can also be done after mechanical thrombectomy including suction thrombectomy is performed with any known devices. As with the application of the thrombolytic agent 18, the contrast material 22 may be applied without worrying about the dislodgement of the clot 38 and migration of the dislodged clot 38 to the arterial system since the inflated distal balloon 16 is blocking entry of the dislodged clot 38 into the artery.

Once the graft is visualized using the contrast material 22, the inflated distal balloon 16 can be pulled back toward the venous side 46, eradicating the arterial plug and removing the platelet plug and residual arterial anastomosis from the shunt-artery juncture. This can be repeated several times if needed. If necessary, direct injection of thrombolytic agent can be performed also towards the venous anastomosis.

The final step is that of removing the balloon, wire and the sheath.

While the preferred embodiments have been shown and described, it will be understood that there is no intent to limit the invention by such disclosure, but rather, is intended to cover all modifications and alternate constructions falling within the spirit and scope of the invention.

1. A catheter, comprising:
a catheter body with a compliant balloon secured at a distal end of the catheter body, the balloon including a proximal end and a distal end; and
at least one infusion aperture directly adjacent the proximal end of the balloon.

2. The catheter according to claim 1, wherein the catheter body includes a first lumen in fluid communication with the interior of the balloon allowing the balloon to be inflated and deflated under the control of a syringe coupled to the first lumen and a second lumen maintained in fluid communication with the at least one infusion aperture located directly adjacent the proximal end of the balloon.

3. The catheter according to claim 1, wherein the balloon is latex.

4. The catheter according to claim 1, wherein the balloon includes an elongated body having a distal portion adjacent the distal end of the balloon and a proximal portion adjacent to the proximal end of the balloon, and compliance of the balloon changes from its distal end to its proximal end.

5. The catheter according to claim 4, wherein the distal portion of the balloon is more compliant than the proximal portion of the balloon and, therefore, inflates first.

6. The catheter according to claim 5, wherein the balloon is latex.

7. The catheter according to claim 5, wherein the balloon is substantially cylindrical with a constant diameter along its length.

8. The catheter according to claim 5, wherein the balloon has a length of approximately 3 cm.

9. The catheter according to claim 5, wherein the balloon is hourglass shaped.

10. The catheter according to claim 5, wherein the balloon is frustoconically shaped, and the proximal portion of the balloon has a larger diameter than the distal portion of the balloon.

11. The catheter according to claim 5, wherein the proximal end of the balloon is designed to extend proximally and slightly cover the infusion apertures.

12. The catheter according to claim 5, further including a catheter tip provided at the distal end of the catheter body at a position distal to the distal end of the balloon, wherein the catheter tip is provided with a relatively sharp but soft distal end and includes a hydrophilic surface.

13. The catheter according to claim 12, wherein the catheter tip includes a taper optimizing reduced drag as the catheter tip is moved through a clot.

14. The catheter according to claim 1, wherein the balloon is substantially cylindrical with a constant diameter along its length.

15. The catheter according to claim 1, wherein the balloon has a length of 3 cm.

16. The catheter according to claim 1, wherein the balloon is hourglass shaped.

17. The catheter according to claim 1, wherein the balloon is frustoconically shaped, and the proximal portion of the balloon has a larger diameter than the distal portion of the balloon.

18. The catheter according to claim 1, wherein the proximal end of the balloon is designed to extend proximally and slightly cover the infusion apertures.

19. The catheter according to claim 1, further including a catheter tip provided at the distal end of the catheter at a position distal to the distal end of the balloon, wherein the catheter tip is provided with a relatively sharp distal end and includes a hydrophilic surface.

20. The catheter according to claim 19, wherein the catheter tip includes a taper optimizing reduced drag as the catheter tip is moved through a clot.

21. The catheter according to claim 19, wherein the catheter tip is curved allowing steering across an arterial anastomosis.

22. The catheter according to claim 1, further including two infusion apertures that are diametrically opposed.

23. The catheter according to claim 1, wherein the proximal end of the balloon is designed to extend proximally and slightly cover the infusion apertures.

24. The catheter according to claim 1, further including radiopaque markers at the distal end of the catheter body.

25. The catheter according to claim 1, wherein the balloon is radiopaque.

26. The catheter according to claim 1, wherein the at least one infusion aperture is substantially closed until such a time that adequate pressure is applied for opening the at least one infusion aperture.

27. The catheter according to claim 1, wherein the catheter is approximately 40 cm to approximately 60 cm long.
